(19) United States
(12) Patent Application Publication
(10) Pub. No.: US 2011/0125016 A1
(43) Pub. Date: May 26, 2011

(54) FETAL RENDERING IN MEDICAL DIAGNOSTIC ULTRASOUND

(75) Inventors: Roee Lazebnik, San Jose, CA (US); Gareth Funka-Lea, Cranbury, NJ (US)

(73) Assignee: SIEMENS MEDICAL SOLUTIONS USA, INC., Malvern, PA (US)

(21) Appl. No.: 12/625,867

(22) Filed: Nov. 25, 2009

(51) Int. Cl. A61B 8/14; G06K 9/00; G06T 15/00

(52) U.S. Cl. 600/443; 382/131; 345/419

(57) ABSTRACT

A fetal skeleton is rendered with medical diagnostic ultrasound. Ultrasound scans of fetal skeleton may acquire data at a rate sufficient to avoid some fetal movement artifacts as compared to magnetic resonance or computed tomography. To better visualize the fetal skeleton, the ultrasound data is used to segment the fetal bone from tissue. By extracting this information, a skeleton in three dimensions is determined. Information representing internal bone locations may be used for fetal bone imaging. Without repeating the segmentation and without adjustments for volume thickness, the skeleton may be visualized from different orientations. A volumetric or surface rendering is performed, allowing addition of lighting queues not available with MIP or other projection rendering free of segmentation. The lighting queues may better indicate actual size and orientation of bones relative to each other on the rendered image.

### BACKGROUND

The present embodiments relate to ultrasound imaging of a fetus. In particular, images of a fetal skeleton are generated using ultrasound.

Skeletal dysplasias are a heterogeneous group of conditions associated with abnormalities of the skeleton, including abnormalities of bone shape, size, and density. The skeletal dysplasias manifest as abnormalities of the limbs, chest, or skull. The prevalence of skeletal dysplasias (excluding limb amputations) is estimated at 2.4/10,000 births and overall prevalence among perinatal deaths is 9.1/1000. If suspected during a routine obstetrical two-dimensional ultrasound examination, then a more detailed ultrasound-based survey is recommended.

For more detailed surveys, fetal skeletal visualization is predominately performed using two-dimensional ultrasound. Studies have established the utility of a volumetric (three-dimensional) approach. Compared with two-dimensional imaging, a volumetric approach enables the clinician to more intuitively visualize skeletal structures as well as relationships between adjacent structures.

Using sonographic imaging, there is typically significant echogenicity difference between fetal bone and soft tissue. Specifically, bone is hyperechoic relative to surrounding soft tissue. For adults and children, the difference may be such that volumetric imaging is not possible due to shadowing. For fetal bone, the bone density may allow for volumetric imaging. Due to the high contrast difference, the common volumetric rendering method for visualizing bony structures is maximum intensity projection (MIP). MIP depicts a slab of tissue (volume) as a two-dimensional image by only displaying the most intense (echogenic) voxel value encountered along projected paths perpendicular to the image plane. Thus, an echogenic bony structure contained within the slab is visualized on the resulting image even if surrounded by soft tissue. The most significant advantage of this approach is the relatively easy visualization of bony structures. FIG. 1 shows an example MIP rendering of fetal bone.

There are limitations to MIP-based visualization. First, adjacent bony structures contained within a given volume and along the same projected path cannot be differentiated. Thus, there is a tradeoff between contrast and spatial resolution, which can be manipulated by adjusting the thickness of the volume. Second, apparent foreshortening of structures occurs if they are not parallel to the image plane. In addition, there are no visual cues that foreshortening occurs, so the user must often examine a given structure using multiple orientations to gauge its true shape. For each orientation, the MIP process is repeated. Third, true volume-based measurements including distances are not possible. Lastly, the adjustment of the volume orientation and thickness for rendering may be difficult to optimize, requiring multiple renderings with the eventual result less than desired.

### BRIEF SUMMARY

By way of introduction, the preferred embodiments described below include a method, system, instructions, and computer readable media for fetal rendering in medical diagnostic ultrasound. Ultrasound scans of fetal skeleton may acquire data at a rate sufficient to avoid some fetal movement artifacts as compared to magnetic resonance or computed tomography. To better visualize the fetal skeleton, the ultrasound data is used to segment the fetal bone from tissue. By extracting this information, a skeleton in three dimensions is determined. Information representing internal bone locations (e.g., full thickness of bone) may be used for fetal bone imaging. Without repeating the segmentation and without adjustments for volume thickness, the skeleton may be visualized from different orientations. A volumetric or surface rendering is performed, allowing addition of lighting queues not available with MIP or other projection rendering free of segmentation. The lighting queues may better indicate actual size and orientation of bones relative to each other on the rendered image.

In a first aspect, a method for fetal rendering in medical diagnostic ultrasound is provided. Ultrasound data representing a volume including a fetus is acquired. The fetus has a skeleton and tissue, and the ultrasound data represents acoustic echoes from the skeleton and the tissue. The skeleton of the fetus represented by the ultrasound data is segmented from tissue represented by the ultrasound data. The segmenting is performed using the ultrasound data. An image is rendered from the ultrasound data representing at least the skeleton. The rendering is a function of a surface of the skeleton where the surface is determined from the segmentation.

In a second aspect, a system is provided for fetal rendering in medical diagnostic ultrasound. An ultrasound imaging system is configured to scan an internal volume of a patient with a transducer positioned adjacent to the internal volume. A processor is configured to determine locations corresponding to fetal bone from ultrasound information acquired by the ultrasound imaging system through the scan. The determination is a function of a size parameter, a shape parameter, or both the size and shape parameters. The processor is configured to generate a three-dimensional rendering from the ultrasound information where the generation is a function of the locations corresponding to fetal bone. A display is operable to generate an image of the three-dimensional rendering. The image represents a skeleton of a fetus.

In a third aspect, a computer readable storage medium has stored therein data representing instructions executable by a programmed processor for fetal rendering in medical diagnostic ultrasound. The storage medium includes instructions for extracting first locations associated with fetal skeleton from second locations representing the fetal skeleton and soft tissue, the extracting being from ultrasound data representing the second locations, and generating a visualization from the ultrasound data as a function of the first locations, the visualization including lighting queues that are a function of the first locations.

In a fourth aspect, a method is provided for fetal rendering in medical diagnostic ultrasound. Ultrasound data representing a volume including a fetus is acquired. The fetus has a skeleton where the ultrasound data represents acoustic echoes from the skeleton, including locations on a surface of the skeleton and locations interior to a bone of the skeleton. An image is rendered from the ultrasound data representing at least the skeleton. The rendering is a function of the surface of the skeleton and the ultrasound data representing the locations interior to the bone of the skeleton.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is an example medical image of a fetus rendered with maximum intensity projection;

FIG. 2 is a flow chart diagram of one embodiment of a method for fetal rendering in medical diagnostic ultrasound;

FIG. 3 is an example medical image of a fetus rendered from segmented fetal skeleton information; and

FIG. 4 is a block diagram of one embodiment of an ultrasound system for fetal rendering in medical diagnostic ultrasound.

### DETAILED DESCRIPTION OF THE DRAWINGS AND PRESENTLY PREFERRED EMBODIMENTS

Fetal ultrasound is the gold standard for pre-natal detection and diagnosis of skeletal dysplasias. While volume-based imaging has significant advantages compared with conventional two-dimensional imaging, MIP volume-visualization methods require manual adjustment of parameters and demonstrate other significant limitations. An automated method for volume-based visualization of the fetal skeleton using obstetric sonographic data is provided. The method utilizes the hyperechogenicity of the fetal skeleton relative to adjacent soft tissue structures to automatically segment bony structures prior to volumetric or surface rendering. Both heuristic and image-based knowledge are used to segment the skeleton for volumetric or surface rendering.

The method may be implemented on and/or with any imaging system or workstation. In one embodiment, skeletal rendering is attractive for advanced (level 2) OB imaging centers in diagnosis of genetic and other fetal skeletal pathologies using high-end volume imaging equipment. Other locations or facilities and equipment may be used. In one embodiment, technology for the OB market segment using the ACUSON 52000 implements the scanning, segmentation, and rendering. One or more, such as four, wobbler or other volume scanning transducers are used to scan all or part of a fetus.

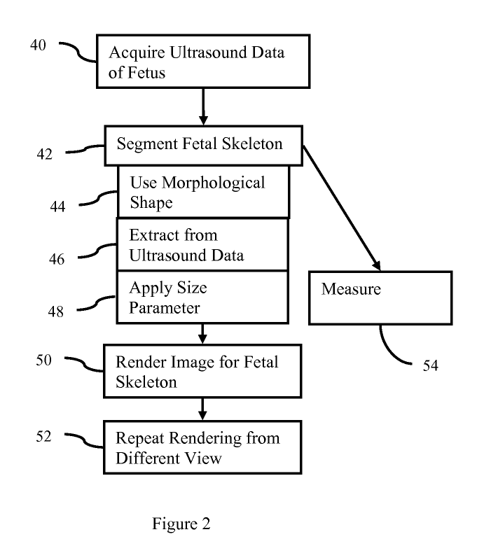

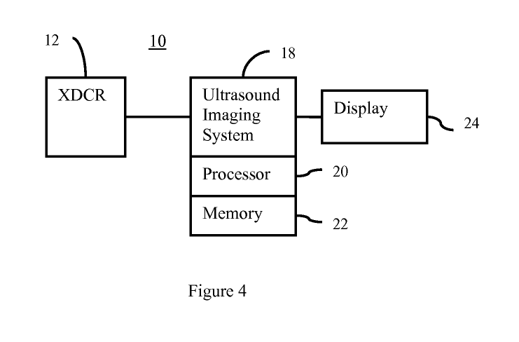

FIG. 2 shows a method for fetal rendering in medical diagnostic ultrasound. The acts of FIG. 2 are implemented by the system 10 of FIG. 4 or a different system. The acts shown in FIG. 2 are performed in the order shown or a different order. Additional, different, or fewer acts may be performed. For example, acts 52 and/or 54 may not be used.

In act 40, ultrasound data representing a volume including a fetus is acquired. The fetus has a skeleton and tissue. Acoustic energy echoes from the skeleton and tissue and is received by a transducer. The resulting ultrasound data represents the acoustic echoes from the skeleton and the tissue. The ultrasound data may also include echoes from tissue of the pregnant female, such as tissue between the transducer and the fetus or tissue around the fetus.

Since fetal bone may be less dense than adult bone, acoustic echoes may be received from within the bone. The entire fetal bone, including the bone surface and interior bone, may be scanned. The resulting ultrasound data represents the surface and the interior portions of the fetal bone. Since the acoustic energy may penetrate into the bone, the resulting ultrasound data and imaging may represent internal bone structure.

The scanning may be for B-mode, color flow mode, tissue harmonic mode, contrast agent mode or other now known or later developed ultrasound imaging modes. Combinations of modes may be used, such as scanning for B-mode and Doppler mode data. Any ultrasound scan format may be used, such as a linear, sector, or Vector®. Using beamforming or other processes, data representing the scanned region is acquired. The data is in an acquisition format (e.g., Polar coordinate system) or interpolated to another format, such as a regular three-dimensional grid (e.g., Cartesian coordinate system). Different ultrasound values represent different locations within the volume.

Any type of scanning may be used, such as planar or volume scanning. For planar scanning, multiple planes are sequentially scanned. The transducer array may be rocked, rotated, translated or otherwise moved to scan the different planes from the same acoustic window or multiple acoustic windows. The volume is scanned by electronic, mechanical, or both electronic and mechanical scanning. The resulting data represents a volume.

The same region may be scanned multiple times from the same acoustic window. The resulting data is combined, such as by persistence filtering, a more optimal one of the resulting data sets is selected, or an on-going or real-time sequence of images are generated from the multiple scans.

In one embodiment, the scanning is from different acoustic windows. Any two or more different acoustic windows or transducer locations may be used so that an extended volume (larger than possible by one array at one acoustic window) is acquired. The transducer is sequentially positioned at different windows. Alternatively, multiple transducers are used to allow either sequential or simultaneous scanning from different windows.

In another embodiment, the ultrasound data is acquired by data transfer or from storage. For example, ultrasound data from a previously performed ultrasound examination is acquired from a picture archival or other data repository. As another example, ultrasound data from an on-going examination or previous examination is transferred over a network from one location to another location, such as from an ultrasound imaging system to a workstation in the same or different facility.

In act 42, the skeleton of the fetus represented by the ultrasound data is segmented from tissue represented by the ultrasound data. In order to enhance the fetal skeleton within a sonographic volume, every location within the volume is classified as to whether the location is more likely to represent skeleton or something other than skeleton. Locations associated with fetal skeleton are extracted from all the locations. The segmentation distinguishes between locations for fetal skeleton and locations for soft tissue or other structure. The soft tissue may be fetal or tissue of the pregnant female.

Any now known or later developed segmentation may be used. For example, the segmentation may be based on region growing, gradients, template matching, rigid or non-rigid transformation, or border detection. Masking may be used, such as to remove locations associated with the tissue of the pregnant female. Filtering may be used to remove or reduce noise or artifacts. For example, a median filter is applied to remove speckle. Any image segmentation or classification into two classes for the purpose of enhancing the fetal skeleton for volumetric rendering may be used.

The segmentation is automatic. The user activates the segmentation, such as by selecting an input data set and a fetal skeleton rendering application. The segmentation and/or rendering occur without further user input. Alternatively, the user inputs one or more parameters, such as placing seeds indicating a location of skeleton and/or seeds indicating a location of soft tissue. Other user inputs to assist with semi-automatic segmentation may be provided. In yet other embodiments, the segmentation is manual. The user traces or otherwise delineates the skeleton.

In one embodiment, the relative hyperechogenicity of the fetal skeleton relative to surrounding structures or tissue is used for segmentation. Other heuristic parameters may be used in the segmentation, such as the spatial relationship between anatomically continuous skeletal structures and assumptions of maximum and minimum sizes of skeletal elements. Using none, one, both, and/or additional assumptions or parameters, the fetal skeleton is segmented from the surrounding soft tissues.

Acts 44, 46, and 48 represent one example embodiment for segmentation. The locations associated with the skeleton are extracted based on size, shape, or size and shape as indicated by the ultrasound data. None, only one, only two, all three, or combinations thereof with additional acts may be used.

The size or shape may be used at any point in the segmentation process. For example, the ultrasound data is filtered in preparation for labeling locations as skeleton or not. The filtering uses a kernel and/or type of filter adapted to enhance structure of particular sizes and/or shapes. The same data may be separately filtered or filtering in parallel to emphasize different sizes or shapes in each resulting set. Alternatively, the data is sequentially filtered or only filtered once. As another example, size and/or shape criteria are applied to the locations labeled as skeleton. If the skeletal locations do not satisfy the criteria, then the locations are relabeled as not skeleton or as tissue.

An example additional act is removal or reduction of values for locations associated with the abdomen. The abdomen tissue of the pregnant female may include relatively bright structures, such as the diaphragm. This tissue is removed by masking. Any masking may be used, such as manual tracing or border detection.

In one embodiment, a random walker segmentation identifies locations associated with abdomen tissue. For example, one of the segmentations identified in U.S. Published Application Nos. 20050163375, 20050226506, 20060050959, 20060147115, or 20060147126 or subsequent improvements is used. Seeds are placed by a user or by a processor. Seeds may be placed adjacent to the transducer location, such as within 1-3 cm to identify starting locations for abdomen tissue and other seeds placed in a center region of the volume (e.g., seeds in a wedge pattern from ⅓ to ⅔ of the range dimension centered laterally). The seeds designate abdomen tissue and fetus. A probability field is determined. A walker is simulated progressing from each seed, but is biased (e.g., spring function) by the data to wander along uniform intensity and avoid intensity variation. A potential field is created, and a threshold is applied to distinguish the regions. For example, a 50% threshold is applied such that a probability of fetus greater than 50% is selected as the fetus and other regions as tissue. Other thresholds may be used.

The segmented locations associated with the fetus and the corresponding ultrasound values are used for further segmentation of the fetal skeleton. In act 44, the segmenting is performed as a function of a morphological shape associated with the skeleton. The skeleton has a distinct morphology. Bones tend to have plate like, knobby, or elongated thin structure. The skeleton forms elongated structures such as ribs and limb bones, knobs such as the head of the femur, and curved plates such as the skull or pelvis.

The ultrasound data is filtered prior to labeling the locations as skeletal or not. The filtering is to enhance the skeleton relative to the tissue. The filtering is a function of the morphological shape. A top hat or other filter identifies bright (higher intensity) regions with bone like shapes (e.g., long and thin bright region). The white top hat filter takes the difference of the original image and a version of the image on which an open transform has been performed. The open transform is a filter that dilates an erosion of the original image. Other filters or combinations of filters may be used. In other embodiments, no filtering is provided.

In act 46, the segmenting is further performed using the ultrasound data. The values of the ultrasound data at the fetal locations are used to distinguish the fetal skeleton from other tissue. The values are the filtered values, but may be unfiltered values. The locations associated with skeleton are extracted from locations representing the fetus, both skeleton and tissue. Several characteristic features are considered in making the segmentation into the two classes “skeleton” (bone) and “not-skeleton” (not-bone). Calcified bone is bright (hyperechoic) and if one location is bone then near-by locations with bright intensities are also likely to be bone. The ultrasound data provides intensity values indicating relative brightness.

Any segmentation, now known or later developed, may be used. For example, the random walker approach is used. In one embodiment, an adaptive threshold approach is used. The adaptive threshold is applied to the filtered data to distinguish between tissue and skeleton. Different threshold or intensity values are selected. For each possible threshold, a variance of the data above the threshold and a variance of the data below the threshold are calculated. The threshold value associated with the minimum of the sum of these two variances is selected. The search range for the threshold may be limited, such as at 20% change from an average value for the entire region. Other adaptive or non-adaptive threshold approaches may be used.

The thresholding is applied to all of the fetal locations. Alternatively, different adaptive thresholds are applied to different sub-sets of locations, such as sub-volumes at different depths having different adaptive thresholds. The resulting adaptive thresholds are applied separately or averaged and applied to the whole.

Locations associated with ultrasound values above the threshold are labeled as skeleton, and other locations are labeled as other structure. Values equal to the threshold are labeled as skeleton or other structure.

In act 48, the expected size of bone structure is used for the segmenting. Noise, such as speckle, or other artifacts may be mislabeled as bone. The output locations labeled as skeleton are tested. To remove the misidentified locations, the locations labeled as bone are grouped. Any continuous region of bone locations is formed as a group. The size of each group is compared to a size and/or shape criteria. A criterion may be a volume, such as a minimum and maximum volume for bone. Any minimum or maximum value may be used, such as empirically determined values. For example, any group more than ¼ of the total fetal volume is treated as being mislabeled. As another example, any group of less than 9 voxels is treated as being mislabeled. A minimum criterion may be used with the maximum criteria or vice versa.

Other tests or operations may be used to avoid mislabeling or to test the segmentation. For example, the bone locations may be filtered, such as smoothing to relabel one or a small number of locations labeled as tissue but surrounded by bone as bone or to relabel one or a small number of locations labeled as bone but surrounded by tissue as tissue. A binary filter is applied where tissue locations are assigned zero values and bone locations assigned one values. The low pass filter removes small bone and small tissue locations by application to the binary data.

The result from the segmentation is identification of locations associated with fetal skeleton. The ultrasound data for the skeleton locations is the same or different than input to the segmentation process. For example, the ultrasound data after filtering to enhance bone is used. Other processes may be used to alter the values at skeleton, tissue, or both locations. As another example, the ultrasound data acquired for segmentation is used. The segmentation outputs the locations associated with skeleton.

In act 50, a visualization is generated from the ultrasound data. The visualization is generated as a function of the bone or skeleton locations. For example, only values at skeleton locations are used for imaging. As another example, the locations are used as part of the rendering from all fetal or all volume locations.

The visualization is an image. One or more images are generated from the ultrasound dataset. For example, an image from any arbitrary plane may be generated from the data representing a volume. Multiplanar reconstruction images may be generated. As another example, volume rendering, surface rendering, or other three-dimensional imaging is provided. In yet another embodiment, projection rendering is provided. Multiple renderings from slightly different viewing directions may be generated for stereoscopic viewing.

In one embodiment, rendering of the data is performed using a true volumetric technique. A surface rendering technique is used instead of a projection rendering. The rendering is free of maximum intensity projection. The actual volume extent of the skeleton is available for rendering rather than identifying a maximum value along a viewing dimension. Instead of reducing the volume information down to a plane of data for generating the image, the skeletal extent in three-dimensions is used to generate the image.

By rendering based on skeletal locations, the user may more intuitively manipulate the skeleton in three dimensions. In one embodiment, any amnioscopic rendering is used. Other rendering methods may be utilized to visualize the data.

In one embodiment of visualization, the ultrasound data from the segmented skeleton is emphasized relative the ultrasound data of the tissue prior to rendering. A filter may be applied. Alternatively, weighting is applied. Any value weights may be used, such as values in the range 0.00-1.00. For example, the ultrasound data associated with skeleton locations is weighted by 1.00 or not weighted, and ultrasound data associated with tissue locations is weighted by less than unity, such as 25%. The locations that do not contain skeleton have their intensity value decreased relative to those locations that contain skeleton. Alternatively or additionally, the data for skeletal locations is weighed by weights greater than unity.

The ultrasound data, adjusted or not, represents a volume including at least a portion of the fetal skeleton. The data represents locations within the volume. An image is rendered from the volume data. In one embodiment using the skeletal locations within the volume, a surface rendering is performed. The segmented skeleton locations are used to determine an outer surface of the skeleton. For example, a gradient is determined for each location. The locations associated with a sufficient gradient indicate a transition from skeleton to tissue (i.e., the skeletal surface). Since the ultrasound data may represent internal bone locations, the gradient may be used. Alternatively, the outer surface of the skeleton is identified from the skeleton locations. The surface of the skeleton is rendered. Any now known or later developed surface rendering may be used.

In another embodiment, a volume rendering is performed. A projection rendering is provided, but with averaging, alpha blending, combination, or selection of information from different depths along the viewing direction. The maximum value is not always selected. For example, the value for the first location or connected locations along a viewing direction greater than a threshold is selected for a pixel. Opacity or transparency may be used as part of the rendering. In one embodiment, the data for locations associated with the skeleton is used for the volume rendering and not data for other locations. In another embodiment, the data for locations associated with the skeleton and data for tissue locations within a distance to skeleton locations are used. Transparency or opacity may be used to emphasize the data for skeletal locations relative to tissue locations.

Since the ultrasound data is responsive to echoes from the full thickness of the fetal bone or at least a portion of the interior of the bone, transparency or opacity may be used to provide depth for rendering the skeleton. The data for the skeletal surface or all skeletal locations is transparent or not fully opaque. The rendering is performed with some transparency of the skeleton. For example, the first few voxels of depth are not entirely opaque, but voxels deeper in the bone are set as fully opaque. Where a light source is provided, the light appears to penetrate at least part of the skeleton. Data from two or more locations contributes to a given pixel value.

The visualization optionally includes rendering with a light source. The skeleton is rendered with shading. The shading emulates a light source. The light source is positioned at a different angle relative to the volume than the viewer. Given the skeleton's three-dimensional shape, shadows are cast. The shading greys out or changes the resulting pixel values where portions of the skeleton block the light, at least partially. These lighting queues indicate depth or relative positioning in three-dimensions. The skeleton locations relative to each other and the light source are used to determine the locations associated with shadow. Any now known or later developed shading operation may be used in the rendering.

Shading uses the three-dimensional location of the skeleton. Shading used with any type of rendering provides for rendering as a function of the skeletal locations in a volume. The light source and volume may be positioned automatically or by the user in any arbitrary location.

Surface rendering, volume rendering, and/or rendering with shading may provide advantages over maximum intensity projection rendering. First, adjacent bony structures may be differentiated by simply rotating the volume to visualize their spatial relationship. Second, spatial resolution is fixed by the volume acquisition method, not its processing. Maximum intensity uses a volume thickness setting that may result in loss of resolution. Third, while there is opportunity for the user to vary parameters to control the segmentation and rendering performance, many cases are feasible without user interaction, using a default set of parameters. Fourth, the apparent foreshortening of structures not parallel to the image plane that happens with MIP does not occur or is reduced in affect.

Other than shading, the image may be enhanced by color mapping. The data for locations associated with skeleton are mapped to bone colors, such as ivory. The data for locations associated with tissue are mapped to tissue colors, such as beiges or browns. When rendered with transparency or opacity settings, the resulting visualization appears more natural. The more opaque bone is emphasized relative to the more transparent tissue, but some tissue is still shown.

In act 52, the rendering is repeated. The volume is rendered from a different direction and/or with different rendering settings. To render again, the same segmentation results may be used. The segmentation identifies skeletal locations for the volume. Regardless of the viewing direction, the same locations are associated with the skeleton. The segmentation does not need to be repeated, but may be repeated.

By varying the viewing angle, the resulting images may indicate bone extent to the user. By varying the location of the light source relative to the volume, the resulting images may indicate bone extent to the user. The actual volume extent of the skeleton is reflected in the images.

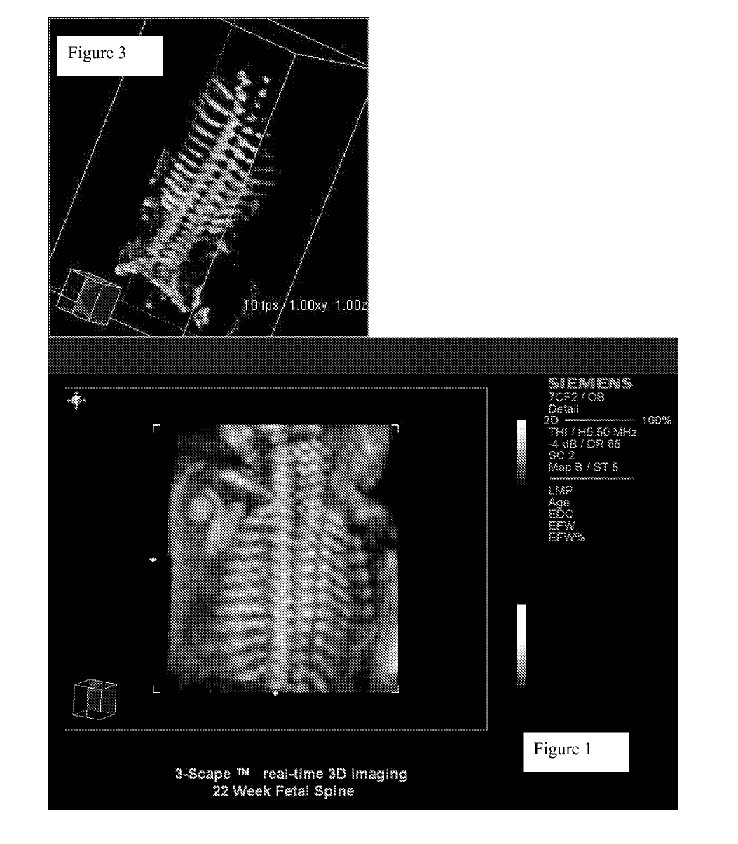

FIG. 3 shows an example rendering of a fetal skeleton using segmentation. The rendering uses a light source for generating shadows. A volume rendering using transparency and tissue information adjacent to the skeleton is provided. In a color image, the bone would be mapped to ivory colors, and the tissue mapped to red or brown colors. The removal of tissue locations spaced from the skeleton avoids indistinct bone rendering, such as compared with FIG. 1.

In act 54, measurements are performed using the skeletal locations. Since the actual spatial extent of the skeleton is determined by segmentation, measurements of the skeleton may be provided. The scan parameters are used to determine the size of each voxel or the spacing between voxels. The user indicates a location for a volume or distance measurement. For a volume measurement, the volume for voxels associated with a selected bone (i.e., connected bone voxels) is determined. For distances, two points are placed. Each point is placed multiple times from different viewing directions to determine the three-dimensional location of the point. Alternatively, the point is placed by the user indicating a direction for the point. The skeleton location on the surface intersected by the direction line is selected as the point. Any now known or later developed technique for indication measurement locations in three-dimensions may be used.

FIG. 4 shows a system 10 for fetal rendering in medical diagnostic ultrasound. The system 10 includes a transducer 12, an ultrasound imaging system 18, a processor 20, a memory 22, and a display 24. Additional, different, or fewer components may be provided. For example, the system 10 includes a user interface. In one embodiment, the system 10 is a medical diagnostic ultrasound imaging system. In other embodiments, the processor 20 and/or memory 22 are part of a workstation or computer different or separate from the ultrasound imaging system 18. The workstation is adjacent to or remote from the ultrasound imaging system 18.

The transducer 12 is a single element transducer, a linear array, a curved linear array, a phased array, a 1.5 dimensional array, a two-dimensional array, a radial array, an annular array, a multidimensional array, a wobbler, or other now known or later developed array of elements. The elements are piezoelectric or capacitive materials or structures. In one embodiment, the transducer 12 is adapted for use external to the patient, such as including a hand held housing or a housing for mounting to an external structure. More than one array may be provided, such as a support arm for positioning two or more (e.g., four) wobbler transducers adjacent to a patient (e.g., adjacent an abdomen of a pregnant female). The wobblers mechanically and electrically scan and are synchronized to scan the entire fetus and form a composite volume.

The transducer 12 converts between electrical signals and acoustic energy for scanning a region of the patient body. The region of the body scanned is a function of the type of transducer array and position of the transducer 12 relative to the patient. For example, a linear transducer array may scan a rectangular or square, planar region of the body. As another example, a curved linear array may scan a pie shaped region of the body. Scans conforming to other geometrical regions or shapes within the body may be used, such as Vector® scans. The scans are of a two-dimensional plane. Different planes may be scanned by moving the transducer 12, such as by rotation, rocking, and/or translation. A volume is scanned. The volume is scanned by electronic steering alone (e.g., volume scan with a two-dimensional array), or mechanical and electrical steering (e.g., a wobbler array or movement of an array for planar scanning to scan different planes).

The ultrasound imaging system 18 is a medical diagnostic ultrasound system. For example, the ultrasound imaging system 18 includes a transmit beamformer, a receive beamformer, a detector (e.g., B-mode and/or Doppler), a scan converter, and the display 24 or a different display. The ultrasound imaging system 18 connects with the transducer 12, such as through a releasable connector. Transmit signals are generated and provided to the transducer 12. Responsive electrical signals are received from the transducer 12 and processed by the ultrasound imaging system 18.

The ultrasound imaging system 18 causes a scan of an internal region of a patient with the transducer 12 and generates data representing the region as a function of the scanning. The scanned region is adjacent to the transducer 12. For example, the transducer 12 is placed against an abdomen or within a patient to scan a fetus. The data is beamformer channel data, beamformed data, detected data, scan converted data, and/or image data. The data represents anatomy of the region, such as the interior of a fetus and other anatomy.

In another embodiment, the ultrasound imaging system 18 is a workstation or computer for processing ultrasound data. Ultrasound data is acquired using an imaging system connected with the transducer 12 or using an integrated transducer 12 and imaging system. The data at any level of processing (e.g., radio frequency data (e.g., I/Q data), beamformed data, detected data, and/or scan converted data) is output or stored. For example, the data is output to a data archival system or output on a network to an adjacent or remote workstation. The ultrasound imaging system 18 processes the data further for analysis, diagnosis, and/or display.

The processor 20 is one or more general processors, digital signal processors, application specific integrated circuits, field programmable gate arrays, controllers, analog circuits, digital circuits, server, graphics processing units, graphics processors, combinations thereof, network, or other logic devices for segmenting and rendering. A single device is used, but parallel or sequential distributed processing may be used.

The processor 20 is configured by software to segment and/or render. The processor implements the segmentation or rendering acts discussed above. For example, the processor 20 determines locations corresponding to fetal bone. The locations are determined from ultrasound information acquired by the ultrasound imaging system 18 through the scan. The intensity values, gradients of the intensity values or other ultrasound information is filtered and/or processed to determine bone locations.

In one embodiment, the determination is a function of a size parameter, a shape parameter, or both the size and shape parameters. For example, the ultrasound data representing at least a portion of a fetus is filtered. The filtering is directional or otherwise emphasizes bright values adjacent to each other in long, narrow, plate like, or knob like shapes. The filtering assists segmentation to better distinguish between the locations corresponding to the tissue and the skeleton. Alternatively, the filtering distinguishes bone from tissue. The output of the filtering is thresholded to determine locations associated with bone.

Other approaches may be used to segment, such as thresholding as a function of variance. Template matching, such as non-rigid transformation, may be used to identify bone locations.

The resulting locations labeled as bone may be tested, such as using size and/or shape tests. If contiguous bone regions match an expected bone template, are of likely size, have a likely shape, or match another parameter, then the locations are left as indicated as bone. Otherwise, the locations are reassigned to be tissue.

The processor 20 is configured to generate a three-dimensional rendering from the ultrasound information. Any type of rendering may be provided, such as surface rendering, volume rendering, or maximum intensity projection rendering. The generation of the rendering is a function of the locations corresponding to fetal bone. The locations are used to define a surface for surface rendering, to determine shadows for rendering with lighting queues, to define the data to be used or compared for projection rendering, or to determine the relevant locations for volume rendering.

The rendering is from data representing a volume to pixels of an image. The rendering may be generated with or without lighting queues, such as rendering with shadowing from a light source.

By rendering as a function of bone locations, the three-dimensional rendering emphasizes the ultrasound information for the locations corresponding to fetal bone. The fetal bone is emphasized relative to the tissue. Other processes than segmentation may additionally emphasize the data of the fetal bone locations, such as filtering or weighting.

The processor 20 is configured, such as through a user interface, to repeat the generation of the three-dimensional rendering. The relative position of the light source, viewer, or volume may be altered. Other parameters may be changed. The rendering is repeated based on the new parameters. The segmentation may be used without change. The rendering is repeated without changing the locations associated with fetal bone since the locations of the fetal bone are determined in three-dimensions.

The processor 20 may also provide quantification. Input from a user interface or automatic determination of locations is used to define points for a distance, area, or volume. The distance, area, or volume are determined, such as measuring a length of a particular bone (e.g., longest different between points in a contiguous bone region), area (e.g., minimum or maximum cross section of a bone contiguous region), or volume (e.g., volume of a contiguous bone region). Other measurements may be determined.

The memory 22 is a tape, magnetic, optical, hard drive, RAM, buffer or other memory. The memory 22 stores the ultrasound data from one or more scans, at different stages of processing, and/or as a rendered image.

The memory 22 is additionally or alternatively a computer readable storage medium with processing instructions. Data representing instructions executable by the programmed processor 20 is provided for fetal rendering in medical diagnostic ultrasound. The instructions for implementing the processes, methods and/or techniques discussed herein are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU, or system.

The display 24 is a CRT, LCD, projector, plasma, printer, or other display for displaying two-dimensional images or three-dimensional representations or renderings. The display 24 displays ultrasound images as a function of the output image data. The image on the display 24 is output from volume or surface rendering. The image is a three-dimensional rendering and represents a skeleton of a fetus.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (We) claim:

1. A method for fetal rendering in medical diagnostic ultrasound, the method comprising:
acquiring ultrasound data representing a volume including a fetus, the fetus having a skeleton and tissue where the ultrasound data represents acoustic echoes from the skeleton and the tissue;
segmenting the skeleton of the fetus represented by the ultrasound data from tissue represented by the ultrasound data, the segmenting performed using the ultrasound data; and
rendering an image from the ultrasound data representing at least the skeleton, the rendering being a function of a surface of the skeleton, the surface determined from the segmentation.

2. The method of claim 1 wherein acquiring comprises acquiring the ultrasound data with a volume scan, the ultrasound data representing tissue of a pregnant female, and wherein the segmenting comprises segmenting the skeleton from the tissue of the fetus and the tissue of the pregnant female.

3. The method of claim 1 wherein segmenting comprises segmenting as a function of the ultrasound data and as a function of a size.

4. The method of claim 1 wherein segmenting comprises segmenting as a function of a morphological shape associated with the skeleton.

5. The method of claim 4 wherein segmenting comprises:
filtering the ultrasound data to enhance the skeleton relative to the tissue, the filtering being a function of the morphological shape;
applying an adaptive threshold to an output of the filtering, the adaptive threshold distinguishing between locations corresponding to the tissue and skeleton; and
identifying the locations output by the application of the adaptive threshold as skeleton associated with a size, the locations output less than the size being associated with the tissue
wherein rendering comprises more heavily weighting ultrasound data associated with the locations identified as skeleton than locations associated with tissue.

6. The method of claim 1 wherein rendering comprises emphasizing the ultrasound data of the segmented skeleton relative to the ultrasound data of the tissue.

7. The method of claim 1 wherein rendering comprises surface rendering with shading as a function of an emulated light source.

8. The method of claim 1 wherein rendering comprises volume rendering where ultrasound data associated with the surface of the skeleton is rendered with some transparency.

9. The method of claim 1 wherein the rendering comprises rendering free of maximum intensity projection.

10. The method of claim 1 wherein rendering comprises mapping ultrasound data associated with the skeleton to bone colors and mapping ultrasound data associated with tissue to tissue colors.

11. The method of claim 1 further comprising repeating the rendering from a different viewing angle using the same ultrasound data representing at least the skeleton output by the segmenting.

12. A system for fetal rendering in medical diagnostic ultrasound, the system comprising:
a transducer;
an ultrasound imaging system configured to scan an internal volume of a patient with the transducer positioned adjacent to the internal volume;
a processor configured to determine locations corresponding to fetal bone from ultrasound information acquired by the ultrasound imaging system through the scan, the determination being a function of a size parameter, a shape parameter, or both the size and shape parameters, the processor configured to generate a three-dimensional rendering from the ultrasound information where the generation is a function of the locations corresponding to fetal bone; and
a display operable to generate an image of the three-dimensional rendering, the image representing a skeleton of a fetus.

13. The system of claim 12 wherein the processor is configured to filter the ultrasound information to enhance the skeleton relative to tissue, the filtering being a function of the shape parameter, to distinguish between the locations corresponding to the tissue and the skeleton, and to reassign the distinguished locations corresponding to skeleton smaller than the size parameter to tissue.

14. The system of claim 12 wherein the processor is configured to generate the three-dimensional rendering with shadowing from a light source.

15. The system of claim 12 wherein the processor is configured to generate the three-dimensional rendering by emphasizing the ultrasound information of the locations corresponding to fetal bone relative to the ultrasound information of the locations corresponding to the tissue.

16. The system of claim 12 wherein the processor is configured to repeat the generating of the three-dimensional rendering from a different viewing angle using the same ultrasound information and the same locations corresponding to fetal bone.

17. In a computer readable storage medium having stored therein data representing instructions executable by a programmed processor for fetal rendering in medical diagnostic ultrasound, the storage medium comprising instructions for:
extracting first locations associated with fetal skeleton from second locations representing the fetal skeleton and soft tissue, the extracting being from ultrasound data representing the second locations; and
generating a visualization from the ultrasound data as a function of the first locations, the visualization including lighting queues that are a function of the first locations.

18. The computer readable storage medium of claim 17 wherein generating the visualization comprises generating the visualization as a surface rendering.

19. The computer readable storage medium of claim 17 wherein extracting comprises extracting as a function of shape, size, or shape and size.

20. The computer readable storage medium of claim 17 wherein the instructions further comprise spatially measuring as a function of the first locations.

21. A method for fetal rendering in medical diagnostic ultrasound, the method comprising:
acquiring ultrasound data representing a volume including a fetus, the fetus having a skeleton where the ultrasound data represents acoustic echoes from the skeleton, including locations on a surface of the skeleton and locations interior to a bone of the skeleton; and
rendering an image from the ultrasound data representing at least the skeleton, the rendering being a function of the surface of the skeleton and the ultrasound data representing the locations interior to the bone of the skeleton.
